(19) United States
(12) Patent Application Publication
(10) Pub. No.: US 2026/0104638 A1
(43) Pub. Date: Apr. 16, 2026

(54) PFAS-FREE ACID GENERATORS FOR LITHOGRAPHY UNDERLAYERS

(71) Applicant: Brewer Science, Inc., Rolla, MO (US)

(72) Inventors: Linhui Zhu, Rolla, MO (US); Richard Elsworth Daugherty, JR., Bland, MO (US); Jakub Koza, Rolla, MO (US); Pengtao Lu, Suwanee, GA (US); Vandana Krishnamurthy, Rolla, MO (US)

(21) Appl. No.: 19/357,318

(22) Filed: Oct. 14, 2025

Related U.S. Application Data
(60) Provisional application No. 63/707,039, filed on Oct. 14, 2024.

(51) Int. Cl. G03F 7/004; C07C 213/08; C07C 213/10; G03F 7/00; G03F 7/09; G03F 7/11

(52) U.S. Cl. CPC G03F 7/0045; C07C 213/08; C07C 213/10; G03F 7/094; G03F 7/11; G03F 7/70033

(57) ABSTRACT

A method of modifying PFAS-free products to render those products suitable for use in semiconductor lithography processes is provided. The modification method involves forming a dispersion or solution of the product and storing it under cold conditions to effect precipitation of the target compound of the product (e.g., an acid generator). The modified product can then be used in various types of lithography compositions to form a desired layer (e.g., spin-on carbon or other carbon-rich layer, hardmask layer) that will meet stringent lithographic industry requirements for semiconductor device manufacture while minimizing use of PFAS-containing materials.

### CROSS-REFERENCE TO RELATED APPLICATIONS

The present application claims the priority benefit of U.S. Provisional Patent Application Ser. No. 63/707,039, filed Oct. 14, 2024, entitled PFAS-FREE THERMAL ACID GENERATORS FOR LITHOGRAPHY UNDERLAYERS, the entirety of which is incorporated by reference herein.

### BACKGROUND

### Field

The present disclosure relates to materials and methods for fabricating microelectronic structures.

### Description of Related Art

The elimination of per- and polyfluoroalkyl substances (PFAS), commonly referred to as “forever chemicals,” is currently a focus in many industries due to their toxicity and tendency to remain in living organisms and in the environment. The elimination of these materials has been mandated by government regulations in many countries, and many manufacturers are voluntarily removing them from their products either in response to, or in anticipation of, these regulations and/or environmental concerns. The semiconductor industry is one such industry, where PFAS compounds are contained in many materials in the form of polymers, surfactants, and catalysts. Reducing or eliminating these compounds poses a challenge, as the PFAS-free materials must still meet the stringent performance requirements, and many of the currently available materials fail to meet these performance requirements. For example, there are commercially available acid generators that meet the requirements of being PFAS-free, but those acid generators are tailored for use in other industries and have other issues, causing them to fail to meet the performance requirements of the semiconductor industry. There is a need for PFAS-free acid generators that also meet semiconductor manufacturing industry requirements.

### SUMMARY

The present disclosure is broadly concerned with a method of forming a structure. The method comprises applying a composition on a surface of a substrate, or on one or more intermediate layers optionally present on said surface of said substrate, to form a PFAS-free layer. The composition comprises a compound (polymeric, oligomeric, or monomeric) and an acid generator source dispersed or dissolved in a solvent system. The acid generator source comprises a quaternary ammonium catalyst, and a 1.5% solution of the acid generator source comprises less than about 300 ppb metal ions. The PFAS-free layer comprises less than about 0.05% by weight PFAS, and at least one of (I), (II), (III), (IV), or (V) is true:
(I) the PFAS-free layer comprises about 50% by weight to about 99% by weight carbon, and further comprising:
   forming a hardmask layer on the PFAS-free layer;
   optionally forming one or more additional intermediate layers on the hardmask layer; and
   forming a photoresist layer on the one or more additional intermediate layers on the hardmask layer, if present, or on the hardmask layer if no additional intermediate layer is present;
(II) one or more intermediate layers is present and includes an uppermost intermediate layer that comprises a carbon-rich layer that comprises about 50% by weight to about 99% by weight carbon, and further comprising:
   optionally forming one or more additional intermediate layers on the PFAS-free layer; and
   forming a photoresist layer on the one or more additional intermediate layers on the PFAS-free layer, if present, or on the PFAS-free layer if no additional intermediate layer is present;
(III) further comprising:
   (a) (i) forming a carbon-rich layer that comprises about 50% by weight to about 99% by weight carbon on the PFAS-free layer;
      (ii) optionally forming one or more additional intermediate layers on the carbon-rich layer; and
      (iii) forming a photoresist layer on the one or more additional intermediate layers on the carbon-rich layer, if present, or on the carbon-rich layer if no additional intermediate layer is present;
   (b) (i) forming a hardmask layer on the PFAS-free layer;
      (ii) optionally forming one or more additional intermediate layers on the hardmask layer; and
      (iii) forming a photoresist layer on the one or more additional intermediate layers on the hardmask layer, if present, or on the hardmask layer if no additional intermediate layer is present; or
   (c) (i) forming a carbon-rich layer that comprises about 50% by weight to about 99% by weight carbon on the PFAS-free layer;
      (ii) forming a hardmask layer on the carbon-rich layer;
      (iii) optionally forming one or more additional intermediate layers on the hardmask layer; and
      (iv) forming a photoresist layer on the one or more additional intermediate layers on the hardmask layer, if present, or on the hardmask layer if no additional intermediate layer is present;
(IV) the surface of the substrate comprises a pattern comprising a plurality of gaps, and the applying a composition comprises depositing the composition in at least some of the gaps; or
(V) the one or more intermediate layers are present and include one or both of a carbon-rich layer that comprises about 50% by weight to about 99% by weight carbon or a hardmask layer, and further comprising forming a photoresist layer on the PFAS-free layer.

The disclosure is also directed towards a method comprising storing a dispersion or solution of an acid generator source comprising an acid generator, with the storing being carried out at a temperature of about −20° C. to about 25° C. for about 32 hours to about 72 hours so as to generate a precipitate including the acid generator. The precipitate is collected.

### BRIEF DESCRIPTION OF THE DRAWINGS

FIG. 1 shows sublimation performance of Examples 3 and 4, as tested in Example 5;

FIG. 2(A) compares the n value of materials from Examples 3 and 4, as tested in Example 6, showing the values are nearly identical;

FIG. 2(B) compares the k value of materials from Examples 3 and 4 materials, as tested in Example 6, showing the values are nearly identical;

FIG. 3 shows CD vs. dose for the POR sample and material from Example 4, as tested in Example 7;

FIG. 4 shows the SEM results from the POR sample, as tested in Example 7;

FIG. 5 shows the SEM results from the Example 4 material, as tested in Example 7;

FIG. 6(A) compares the n value of the POR material to that of the materials from Examples 3 and 4 during the aging study of Example 8, showing similar values for all three materials;

FIG. 6(B) compares the k value of the POR material to that of the materials from Examples 3 and 4 during the aging study of Example 8, showing similar values for all three materials;

FIG. 7 shows the thickness of a POR material as compared to that of the materials from Examples 3 and 4 during the aging study of Example 8;

FIG. 8 shows the undercut from Example 9;

FIG. 9 shows the thickness loss of the material containing TAG 2689 in Example 10;

FIG. 10 shows the thickness loss of the material containing CXC-1821 in Example 10;

FIG. 11 provides sublimation data for the Example 12 material, as determined in Example 13;

FIG. 12 is a graph of CD vs. dose for the Example 12 material, as determined in Example 14;

FIG. 13 is an SEM image (200 kX; tilt angle of 75 degrees) of features formed using the Example 12 material, as described in Example 14;

FIG. 14 is an SEM image (200 kX; tilt angle of 90 degrees) of the sectioned trilayer stack prepared using the Example 17 material, following the procedures described in Example 18;

FIG. 15 is an SEM image (200 kX; tilt angle of 75 degrees) of the sectioned trilayer stack prepared using the Example 17 material, following the procedures described in Example 18; and

FIG. 16 is the KrF lithography focus-exposure matrix (FEM) obtained in Example 18 (Target CD 160L/300P).

### DETAILED DESCRIPTION

The present disclosure is broadly concerned with methods of modifying commercially available products, such as acid generators, so that those products perform according to semiconductor industry requirements, as well as methods of using those modified products in lithography underlayers.

### Modification Method and Products

Any number of commercially available products can be modified with the method described herein, with acid generators (thermal acid generators or photoacid generators) being one such type of product.

As used herein, per- and polyfluoroalkyl substances (“PFAS”) refers to a compound (be it polymeric, oligomeric, or monomeric) that includes an alkyl having two or more fluorine atoms bonded to the same carbon atom.

In one or more embodiments, the chosen compound (e.g., acid generator) is PFAS-free, i.e., the compound does not include an alkyl that has two or more fluorine atoms bonded to the same carbon atom.

In at least some embodiments, the source of the compound (e.g., the acid generator source) comprises little to no PFAS, i.e., less than about 1% by weight, preferably less than about 0.5% by weight, more preferably less than about 0.05% by weight, and even more preferably about 0% by weight PFAS, based on the weight of the source of the compound taken as 100% by weight. “Source of the compound” refers to the product as purchased or as supplied, which may or may not include other components (intentionally and/or inadvertently) in addition to the compound.

In one or more embodiments, the source of the compound is a commercially available product that comprises an acid generator. One suitable acid generator comprises a superacid catalyst, and preferably a blocked superacid catalyst. As used herein, a “superacid catalyst” is one that will generate or behave as a superacid upon exposure to certain conditions (e.g., exposure to actinic radiation or elevated temperatures, such as 55° C. or higher). A “superacid” is an acid with a strength exceeding that of pure (100%) sulfuric acid.

An example of a superacid catalyst suitable for use in the disclosed methods is a quaternary ammonium catalyst (preferably a blocked quaternary ammonium catalyst). One preferred quaternary ammonium catalyst is a salt whose cationic portion comprises

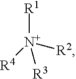

where each of R¹ to R⁴ is individually chosen from alkyls (preferably C₁ to C₄) and aryls (substituted and/or unsubstituted; preferably C₆-C₁₂), with at least one, and preferably two, of R¹ to R⁴ comprising an aryl.

Preferred aryls comprise

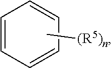

where each R⁵ is individually chosen from —H, alkyls (preferably C₁ to C₄), or alkoxys (preferably C₁ to C₄).

The anionic portion of the salt is preferably chosen to be free of antimony. Suitable such anionic portions include B(C₆F₅)₄— and/or PF₆—.

A preferred quaternary ammonium catalyst for use as an acid generator herein comprises

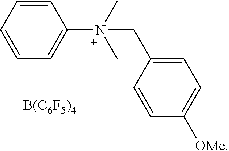 [SMILES: "B(C6F5)4" ; COc1ccc(C[N+](C)(C)c2ccccc2)cc1]

Two commercially available sources of this acid generator are sold under the name K-PURE CXC-1821 by King Industries, TAG-1821 by King Industries, or AA-01 by San-Apro.

The chosen acid generator source preferably comprises less than about 0.1% by weight PFAS, more preferably less than about 0.05% by weight PFAS, and even more preferably about 0% by weight PFAS. Additionally or alternatively, the acid generator source comprises less than about 0.1% by weight antimony, more preferably less than about 0.05% by weight antimony, and even more preferably about 0% by weight antimony. Again, additionally or alternatively, in some embodiments, the acid generator source comprises less than about 0.1% by weight gallium, more preferably less than about 0.05% by weight gallium, and even more preferably about 0% by weight gallium. Percentages by weight are based on the total weight of the acid generator source taken as 100% by weight.

Regardless of the chosen acid generator source, a dispersion or solution of that acid generator source is subjected to the following modification process. Typically, the acid generator source is provided as a solid (e.g., a particulate, such as a powder) material. In such instances, the particulate material should be dispersed or dissolved in a solvent(s) that is selected based on the solubility properties of the particular acid generator source. Typical such solvents include propylene glycol monomethyl ether acetate (PGMEA), propylene glycol monomethyl ether (PGME), propylene glycol ethyl ether (PGEE), acetone, ethyl lactate (EL), propylene carbonate, deionized water, cyclohexanone, γ-butyrolactone (GBL), cyclopentanone, or mixtures thereof.

The acid generator source is typically added to the solvent at a level of about 5% by weight to about 40% by weight, and preferably about 10% by weight to about 20% by weight, based upon the combined weight of the acid generator source and solvent(s) taken as 100% by weight. In some embodiments, deionized water is added to control the temperature of the solution or dispersion. The quantity of deionized water can vary, but the weight ratio of deionized water to solvent(s) is typically about 0.05:1.00 to about 1.00:0.05, preferably about 0.2:1.0 to about 1.0:0.2, more preferably about 0.5:1.0 to about 1.0:0.5, and even more preferably about 1:1.

The acid generator source, solvent, and any deionized water are then mixed to form a substantially homogeneous solution or dispersion. Mixing can be carried out on a roller, for example, with typical mixing times being about 4 hours or longer, preferably about 8 hours to about 24 hours, more preferably about 10 hours to about 15 hours, and even more preferably about 12 hours.

The mixed solution or dispersion is suitably cooled at a temperature of about −20° C. to about 25° C., preferably about −10° C. to about 15° C., and more preferably about 0° C. to about 5° C. Cooling is typically carried out for a time period of about 32 hours to about 72 hours, preferably about 40 hours to about 60 hours, and more preferably about 40 hours to about 50 hours.

After cooling, a precipitate, which was formed during the above-described process and comprises the acid generator, is collected (preferably after filtering) and optionally dried to remove any remaining solvent. Drying can be carried out in a vacuum oven with typical drying temperatures being about 30° C. to about 50° C., preferably about 35° C. to about 45° C., and more preferably about 40° C. Typical drying times are about 12 hours to about 60 hours, preferably about 25 hours to about 50 hours, and more preferably about 35 hours to about 40 hours.

In one or more embodiments, the precipitate comprises little to no PFAS, i.e., less than about 1% by weight, preferably less than about 0.5% by weight, more preferably less than about 0.05% by weight, and even more preferably about 0% by weight PFAS, based on the weight of the precipitate taken as 100% by weight.

Advantageously, in some embodiments an approximately 1.5% solution or dispersion of the precipitate (again, which includes the acid generator) in a solvent (e.g., PGME, PGMEA, PGEE, EL) comprises less than about 300 ppb metal ions, preferably less than about 100 ppb metal ions, more preferably less than about 50 ppb metal ions, and even more preferably less than about 10 ppb metal ions. Metal ion concentrations can be determined as described in Example 11.

In one or more embodiments, an approximately 1.5% solution or dispersion of the precipitate in a solvent (e.g., PGME, PGMEA, PGEE, EL) comprises one or more of the metal ion levels shown in Table A, in any combination.

TABLE A
Metal Ion Concentrations

METAL

| | | | |
|---|---|---|---|
| Na | about 4 ppb or lower | about 3 ppb or lower | about 1.5 ppb or lower |
| Mg | about 1 ppb or lower | about 0.5 ppb or lower | about 0.1 ppb or lower |
| Al | about 0.3 ppb or lower | about 0.2 ppb or lower | about 0.15 ppb or lower |
| K | about 4 ppb or lower | about 2 ppb or lower | about 1 ppb or lower |
| Ca | about 4 ppb or lower | about 2 ppb or lower | about 1 ppb or lower |
| Ti | about 0.04 ppb or lower | about 0.03 ppb or lower | about 0.015 ppb or lower |
| Cr | about 0.3 ppb or lower | about 0.2 ppb or lower | about 0.15 ppb or lower |
| Mn | about 3 ppb or lower | about 2 ppb or lower | about 1 ppb or lower |
| Fe | about 1.2 ppb or lower | about 1 ppb or lower | about 0.6 ppb or lower |
| Ni | about 1 ppb or lower | about 0.5 ppb or lower | about 0.1 ppb or lower |
| Cu | about 1 ppb or lower | about 0.5 ppb or lower | about 0.1 ppb or lower |
| Zn | about 0.8 ppb or lower | about 0.6 ppb or lower | about 0.3 ppb or lower |
| Zr | about 0.6 ppb or lower | about 0.3 ppb or lower | about 0.1 ppb or lower |
| Sn | about 1 ppb or lower | about 0.5 ppb or lower | about 0.2 ppb or lower |
| TOTAL OF | about 24 ppb or lower | about 14 ppb or lower | about 7 ppb or lower |
| ABOVE METALS | | | |

Additionally or alternatively, in some embodiments an approximately 1.5% solution or dispersion of the precipitate in a solvent (e.g., PGME, PGMEA, PGEE, EL) comprises substantially fewer metal ions than an approximately 1.5% solution or dispersion of the unmodified acid generator source (i.e., the starting product before modification) in the same solvent. More particularly, the above-described solution or dispersion of the precipitate comprises about 75% or fewer metal ions, preferably about 85% or fewer metal ions, and more preferably about 90% or fewer metal ions as compared to a solution or dispersion of the unmodified acid generator source at the same concentration. In other words, if a 1.5% solution or dispersion of the unmodified acid generator source includes 100 ppb metal ions, a 1.5% solution or dispersion of the precipitate obtained following the above-described method includes about 25 ppb or fewer metal ions, preferably 15 ppb or fewer metal ions, and more preferably about 10 ppb or fewer metal ions.

Lithography Underlayer Compositions with Modified Acid Generator

It will be appreciated that the above-described modified acid generator can be used in any lithography composition in which an acid generator is needed. This can be accomplished by simply mixing the modified acid generator into the particular composition in place of any other acid generator, and particularly in place of PFAS-containing acid generators.

For example, the modified acid generator can be used in place of thermal acid generators as disclosed in the following commonly-owned patents and published patent applications, the contents of which are each hereby incorporated by reference: U.S. Pat. No. 7,833,692 (Amine-arresting additives for materials used in photolithographic processes); U.S. Pat. No. 8,895,230 (Spin-on carbon compositions for lithographic processing); U.S. Pat. No. 9,496,164 (Cyclic olefin polymer compositions and polysiloxane release layers for use in temporary wafer bonding processes); U.S. Pat. No. 12,024,594 (Multifunctional materials for temporary bonding); US 20220195238 (Chemically homogeneous silicon hardmasks for lithography); US20240030063 (Thermally decomposable fill material); US20230282478 (Coating compositions and methods to enhance SC1 resistance); and US 20240280905 (Underlayer and methods for EUV lithography), all of which are owned by Brewer Science, Inc.

Regardless of the embodiment, the lithography underlayer compositions preferably comprise the above-described modified acid generator and a compound (monomer, oligomer, and/or polymer) dispersed or dissolved in a solvent system. The lithography underlayer composition may also comprise optional ingredients such as those chosen from crosslinkers, surfactants, polymers, catalysts, additives, or mixtures thereof.

Suitable polymers for use as the compound include carbon-rich polymers, such as those chosen from polystyrene, functionalized polystyrene derivatives (e.g., poly(4-methylstyrene), poly(vinyl naphthalene)), polysulfones, polyethersulfones, poly(ether ether ketone), polycarbonates, epoxies, novolacs, polyimides, or combinations thereof.

In some embodiments, suitable polymers and/or oligomers for use as the compound comprise monomers chosen from phenolic compounds, styrene, styrene-containing compounds, glycidyl-containing compounds, 2-hydroxyethyl methacrylate, hydroxypropyl methacrylate, hydroxybutylacrylate, silanes, siloxanes, or combinations thereof.

In the same or different embodiments, the compound can be a monomeric compound, such as those chosen from phenolic compounds, styrene, styrene-containing compounds, glycidyl-containing compounds, 2-hydroxyethyl methacrylate, hydroxypropyl methacrylate, hydroxybutylacrylate, silanes (e.g., 2-(3,4-epoxycyclohexyl)ethyltrimethoxysilane), or combinations thereof.

Additionally, the compound can be functionalized with light absorbing or other moieties (e.g., 9-anthracenecarboxylic acid).

Regardless of the selected compound (i.e., monomer, oligomer, and/or polymer), that compound is typically present in the composition at levels of about 1% by weight to about 30% by weight, more preferably about 3% by weight to about 10% by weight, and even more preferably about 4% by weight to about 7% by weight, based upon the total weight of the composition taken as 100% by weight.

The modified acid generator is preferably present in the composition at levels of about 0.01% by weight to about 10% by weight, more preferably about 0.1% by weight to about 5% by weight, and even more preferably about 0.5% by weight to about 3% by weight, based upon the total weight of the compound (i.e., monomer, oligomer, and or polymer) taken as 100% by weight.

The modified acid generator may be used alone or in conjunction with other non-PFAS-containing acids or acid-generating materials to create the same effect as using the PFAS-containing acid generators. In some embodiments, the composition does not include any acid generators or catalysts other than the modified acid generator (e.g., a modified quaternary ammonium catalyst).

The above ingredients are mixed in a solvent system to form the particular composition. Preferred solvent systems include one or more solvents chosen from PGMEA, PGME, PGEE, cyclopentanone, cyclohexanone, anisole, acetophenone, or mixtures thereof. The solvent system is preferably utilized at a level of about 80% by weight to about 99% by weight, more preferably about 85% by weight to about 97% by weight, and even more preferably about 92% by weight to about 95% by weight, based upon the total weight of the lithography underlayer composition taken as 100% by weight. The material is preferably filtered before use, such as with a 0.1-μm or 0.2-μm PTFE filter.

In embodiments where the modified acid generator is incorporated into a carbon-rich layer (e.g., spin-on carbon layer, or “SOC”), the composition to form that layer preferably comprises about 50% by weight or greater carbon, preferably about 50% by weight to about 99% by weight carbon, preferably about 70% to about 90% by weight carbon, and more preferably about 75% to about 80% by weight carbon, based upon the total solids in the composition taken as 100% by weight.

In one or more embodiments, the composition comprises less than about 0.5%, preferably less than about 0.1%, more preferably less than about 0.01%, and even more preferably about 0% by weight PFAS-containing surfactant, based upon the total weight of the composition taken as 100% by weight. In other embodiments, the composition comprises about 0.001% to about 2% by weight, preferably about 0.001% to about 1% by weight, and more preferably about 0.005% to about 1% by weight PFAS-containing surfactant, based upon the total weight of the composition taken as 100% by weight.

Regardless of the type of composition, it is preferred that the composition is substantially PFAS-free. In other words, preferred embodiments of the composition comprise less than about 2%, preferably less than about 1%, more preferably less than about 0.5%, and even more preferably about 0% by weight PFAS, based upon the total solids in the composition taken as 100% by weight.

### Methods of Using Compositions Including Modified Acid Generator

The above-described compositions can be used to form various types of lithography underlayers, including antireflective coatings, carbon-rich layers, gap-fill layers, planarizing layers, assist layers, and/or adhesion layers. The following generally describes some of these layer-forming processes, with the understanding that the previously described, modified acid generators can be incorporated into any of these layers, as was also explained above with respect to the compositions.

Any microelectronic substrate can be utilized in the disclosed methods, but the substrate is preferably a semiconductor substrate, such as substrates formed from one or more of silicon, SiGe, SiO₂, Si₃N₄, SiON, aluminum, tungsten, tungsten silicide, gallium arsenide, germanium, tantalum, tantalum nitride, Ti₃N₄, hafnium, HfO₂, ruthenium, indium phosphide, tetramethyl silate and tetramethylcyclotetrasiloxane combinations (such as that sold under the name CORAL), SiCOH (such as that sold under the name Black Diamond, by SVM, Santa Clara, CA, US), or glass. Optional intermediate layers may be formed on the substrate prior to processing, with preferred intermediate layers being TiN or SiO₂ layers. The substrate can have a planar surface, or it can include topographic features (via holes, trenches, contact holes, raised features, lines, etc.). As used herein, “topography” refers to the height or depth of a structure in or on a substrate surface.

A layer of lithography underlayer composition is formed on the substrate or any intermediate layers (e.g., a primer layer). Regardless of the lithography underlayer composition utilized, the layer can be formed by any known application method, with one preferred method being spin-coating at speeds of about 1,000 rpm to about 2,000 rpm, and preferably about 1,250 rpm to about 1,750 rpm, for a time period of about 10 seconds to about 90 seconds, and preferably about 30 seconds to 60 seconds. Preferably, the lithography underlayer composition has good spin bowl compatibility, that is, it does not react or form a precipitate with common photoresist solvents such as PGME, PGMEA, ethyl lactate, cyclohexanone, or mixtures thereof.

After the lithography underlayer composition is applied, it is optionally heated to a temperature of about 140° C. to about 230° C., and more preferably about 185° C. to about 215° C., for about 15 seconds to about 90 seconds, and preferably about 30 seconds to about 60 seconds, to evaporate solvents.

As mentioned previously, the modified acid generator can be incorporated into the composition used to form any number of various lithography underlayers. Thus, the foregoing underlayer formation description is optionally repeated for any number of layers, depending on the particular application's needs. For example, antireflective coatings (if included) tend to be one of the lower or first-applied layers, generally closer to the substrate. Carbon-rich layers (if included) tend to be a mid-level layer while hardmask layers (again, if included) tend to be in the upper half of the stack of layers, nearer the photoresist. In applications where both a carbon-rich layer and a hardmask layer are utilized, the hardmask layer tends to be applied to the carbon-rich layer. Assist layers tend to be applied at the upper portion of the stack, often immediately under the photoresist. Adhesion layers find application at any location where two different layers might have properties that are not conducive to adhering to one another (e.g., at the substrate). Gap-fill layers and planarizing layers are typically used once a high topographic region has been created, applied at sufficient levels to fill gaps between features in a pattern, and even to cover those features and form a planar layer on top of those features.

When used as an antireflective coating, the average thickness of the lithography underlayer after baking is typically about 5 nm to about 2.0 μm, and preferably about 75 nm to about 400 nm.

When used as a carbon-rich layer (such as a spin-on carbon layer), the average thickness of the lithography underlayer after baking is typically about 5 nm to about 2.0 μm, and preferably about 75 nm to about 400 nm. Additionally, the carbon-rich layer preferably comprises about 50% by weight or greater carbon, preferably about 50% by weight to about 99% by weight carbon, preferably about 70% to about 90% by weight carbon, and more preferably about 75% to about 80% by weight carbon, based upon the total weight of the layer taken as 100% by weight.

When used as a hardmask layer (e.g., such as the hardmask in a trilayer resist stack), the average thickness of the lithography underlayer after baking is typically about 5 nm to about 1 μm, and preferably about 20 nm to about 50 nm.

When used as gap-fill layer, the average thickness of the lithography underlayer after baking is typically about 5 nm to about 2.0 μm, and preferably about 75 nm to about 400 nm. When used as an assist layer or adhesion layer, the average thickness of the lithography underlayer after baking is typically about 5 nm to about 2,000 nm, and preferably about 75 nm to about 400 nm.

The average thickness is determined by taking the average of thickness measurements at twenty-five different locations of the lithography underlayer, with those thickness measurements being obtained using ellipsometry.

Regardless of the embodiment, any layer including the modified acid generator is preferably substantially PFAS-free. In other words, the formed layer preferably comprises less than about 2% by weight, preferably less than about 1% by weight, more preferably less than about 0.5% by weight, even more preferably less than about 0.05% by weight, and most preferably about 0% by weight PFAS, based upon the weight of that layer taken as 100% by weight.

A photoresist (i.e., imaging layer) can be applied to the lithography underlayer, or to any intermediate layer on the lithography underlayer, to form a photoresist layer. The photoresist layer can be formed by any conventional method, with one preferred method being spin coating the photoresist composition at speeds of about 350 rpm to about 4,000 rpm (preferably about 1,000 rpm to about 2,500 rpm) for a time period of about 10 seconds to about 60 seconds (preferably about 10 seconds to about 30 seconds). The photoresist layer is then optionally post-application baked (“PAB”) at a temperature of at least about 70° C., preferably about 80° C. to about 150° C., and more preferably about 100° C. to about 150° C., for time periods of about 30 seconds to about 120 seconds. The average thickness (determined as described previously) of the photoresist layer after baking is typically about 5 nm to about 120 nm, preferably about 10 nm to about 50 nm, and more preferably about 20 nm to about 40 nm.

The photoresist layer is subsequently patterned by exposure to radiation for a dose of about 10 mJ/cm² to about 200 mJ/cm², preferably about 15 mJ/cm² to about 100 mJ/cm², and more preferably about 20 mJ/cm² to about 50 mJ/cm² at a variety of wavelengths, but preferably about 193 nm or about 13.5 nm (i.e., EUV exposure). More specifically the photoresist layer is exposed using a mask positioned above the surface of the photoresist layer. The mask has areas designed to permit the radiation to reflect from or pass through the mask and contact the surface of the photoresist layer. The remaining portions of the mask are designed to absorb the light to prevent the radiation from contacting the surface of the photoresist layer in certain areas. Those skilled in the art will readily understand that the arrangement of reflecting and absorbing portions is designed based upon the desired pattern to be formed in the photoresist layer and ultimately in the substrate or any intermediate layers.

After exposure, the photoresist layer is preferably subjected to a post-exposure bake (“PEB”) at a temperature of less than about 180° C., preferably about 60° C. to about 140° C., and more preferably about 80° C. to about 130° C., for a time period of about 30 seconds to about 120 seconds (preferably about 30 seconds to about 90 seconds).

The photoresist layer is then dry etched or (wet) developed to form the pattern. Depending upon whether the photoresist used is positive-working or negative-working, the developer either removes the exposed portions of the photoresist layer or removes the unexposed portions of the photoresist layer to form the pattern. The pattern is then transferred through the various layers, and finally to the substrate. This pattern transfer can take place via plasma etching (e.g., CF₄ etchant, O₂ etchant) or a wet etching or developing process.

Additional advantages of the various embodiments will be apparent to those skilled in the art upon review of the disclosure herein and the working examples below. It will be appreciated that the various embodiments described herein are not necessarily mutually exclusive unless otherwise indicated herein. For example, a feature described or depicted in one embodiment may also be included in other embodiments but is not necessarily included. Thus, the present disclosure encompasses a variety of combinations and/or integrations of the specific embodiments described herein.

As used herein, the phrase “and/or,” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination.

The present description also uses numerical ranges to quantify certain parameters relating to various embodiments. It should be understood that when numerical ranges are provided, such ranges are to be construed as providing literal support for claim limitations that only recite the lower value of the range as well as claim limitations that only recite the upper value of the range. For example, a disclosed numerical range of about 10 to about 100 provides literal support for a claim reciting “greater than about 10” (with no upper bounds) and a claim reciting “less than about 100” (with no lower bounds).

### EXAMPLES

The following examples set forth methods in accordance with the disclosure. It is to be understood, however, that these examples are provided by way of illustration, and nothing therein should be taken as a limitation upon the overall scope.

### Example 1

### Modification of K-Pure® CXC-1821 Material

In a 1-L Aicello bottle, 60.32 grams of K-Pure® CXC-1821 (King Industries, Norwalk CT) was dissolved in 180 grams of PGME. Next, 180 grams of deionized water was slowly added to control the temperature of solution. The bottle was mixed on a roller for 12 hours and then was placed into a freezer at 2° C. for two days. The precipitate was filtered and collected followed by drying in a vacuum oven at 40° C. for 38 hours to completely remove all solvents. The yield was 51.7 grams of CXC-1821.

### Comparative Example 1

Coating Formulation with Epoxy Cresol Novolac

In this Example, 4.199 grams of ECN1299 (Brewer Science, Rolla, MO), 0.08 gram of TAG-2689 (King Industries), 0.01 gram of Capstone™ FS-3100 surfactant (Kukudo Chemical Co, Ltd., Korea), 11.96 grams of PGMEA, and 3.75 grams of PGME were added to an Aicello bottle. The bottle was mixed on a roller for 12 hours.

### Example 2

Coating Formulation with

### Epoxy Cresol Novolac and Example 1 Modified Acid Generator

In this Example, 4.01 grams of ECN1299 (Brewer Science, Rolla, MO), 0.04 gram of modified CXC-1821 prepared in Example 1, 0.01 gram of Capstone™ FS-3100 surfactant (Kukudo Chemical Co, Ltd., Korea), 76.75 grams of PGMEA, and 19.19 grams of PGME were added to an Aicello bottle. The bottle was mixed on a roller for 12 hours.

### Example 3

Coating Formulation with

### Epoxy Cresol Novolac and Example 1 Modified Acid Generator

In this Example, 4.01 grams of ECN1299, 0.04 gram of the modified CXC-1821 obtained in Example 1, 0.01 gram of AP-001 surfactant (NOF Corporation, Japan Hyogo), 76.75 grams of PGMEA, and 19.19 grams of PGME were added to an Aicello bottle. The bottle was mixed on a roller for 12 hours.

### Example 4

Coating Formulation with

### Epoxy Cresol Novolac and Example 1 Modified Acid Generator

In this Example, 4.02 grams of ECN1299, 0.04 gram of the modified CXC-1821 obtained in Example 1, 76.75 grams of PGMEA, and 19.19 grams of PGME were added to an Aicello bottle. The bottle was mixed on a roller for 12 hours.

### Example 5

Coat Quality Performance of Materials from Examples 3 and 4

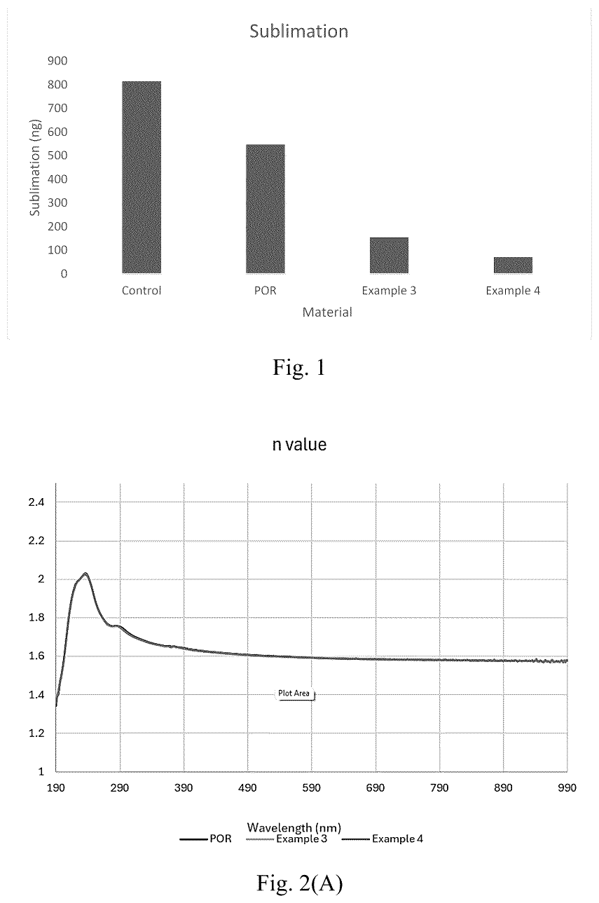

Films were spin coated on 100-mm silicon wafers by spin coating the materials from Examples 3 and 4 at 1,500 rpm and then placed on a hot plate under a QCM (quartz crystal microbalance) to determine the level of sublimation, with the test and control samples being analyzed at the same conditions. Once under the microbalance, the films were exposed to a 205° C. hot plate for 120 seconds, and the sublimation levels were recorded. These sublimation results were compared to a standard, acrylic-based antireflective coating (control; ARC® 29A material, Brewer Science, Inc.) and the spin-on carbon (“POR”) material prepared in Comparative Example 1. This comparison is shown in FIG. 1.

### Example 6

Optical Performance of Materials from Examples 3 and 4

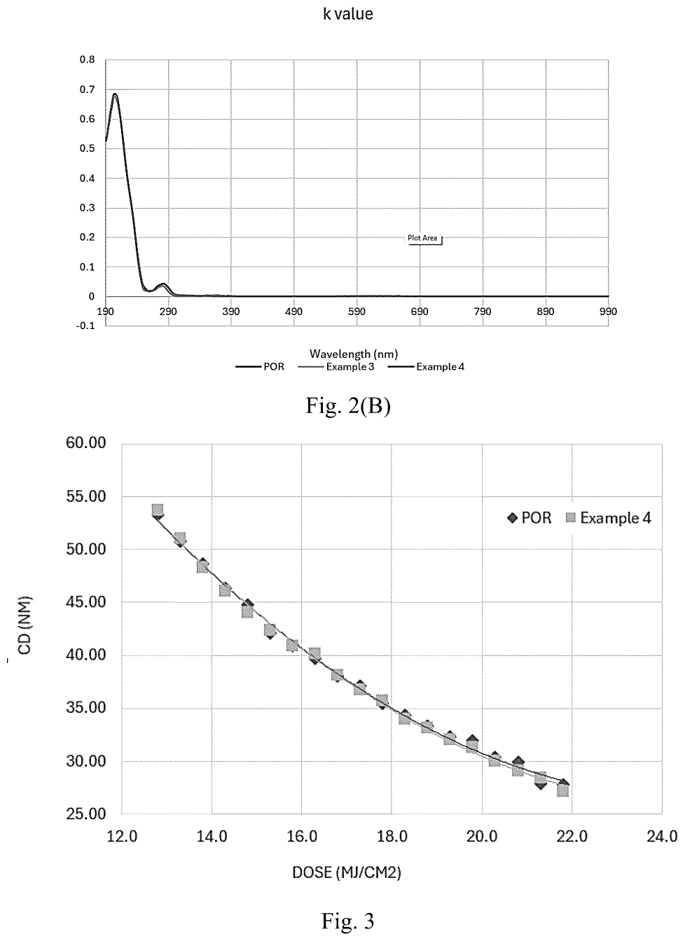

Films were spin coated on 100-mm wafers by spin coating the materials from Examples 3 and 4 at 1,500 rpm and then baked at 205° C. for 60 seconds. The films were then analyzed with a M2000 spectroscopic ellipsometer to obtain the optical properties (i.e., n and k values). These results were compared with a comparable spin-on carbon material (Brewer Science, Inc.). FIG. 2(A) shows the n value, while FIG. 2(B) shows the k value.

### Example 7

### Lithography Evaluation of Example 4 Material

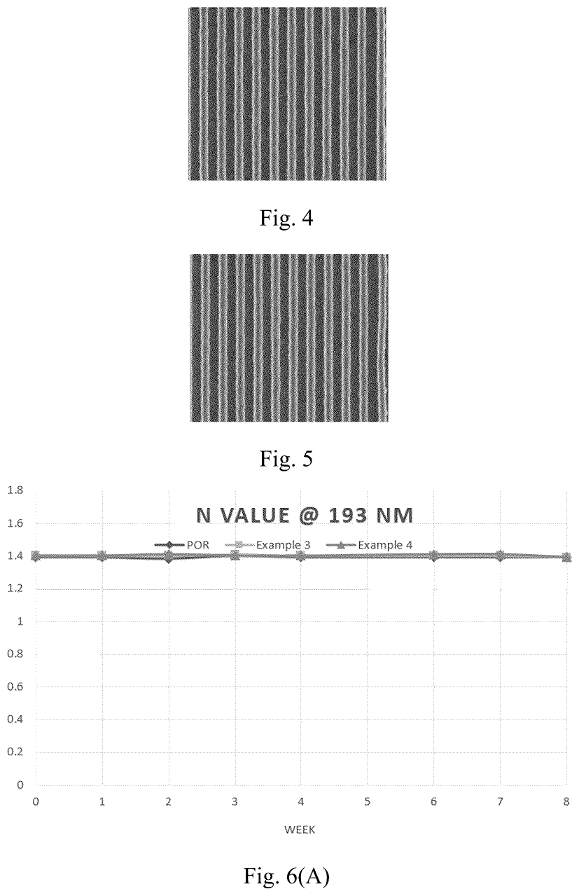

Material from Example 4 was coated on a blank 300-mm Si wafer by spin coating at 1,500 rpm and then baking at 205° C. for 60 seconds. OptiStack® HM825 material (a hardmask available from Brewer Science, Inc.) was then spin coated at 1,500 rpm for 60 seconds on the SOC layer with a 205° C. bake for 60 seconds. A resist (AIM 5484, JSR, Tokyo, Japan) was applied to the Si-HM layer and baked at 120° C. for 60 seconds. Lithography exposure at a wavelength of 193 nm was carried out, and development was performed with OPD5262 (FUJIFILM Electronic Materials, Belgium) at imec. Process windows were then analyzed by top-down CD-SEM analysis and plotted. The results were compared to a standard spin-on carbon material (Brewer Science, Inc.). FIG. 3 compares the CD to dose of the Comparative Example 1 material (“POR”) to the Example 4 material, while FIGS. 4-5 provide SEM images of the POR material (FIG. 4) and the Example 4 material (FIG. 5).

### Example 8

### Aging Studies

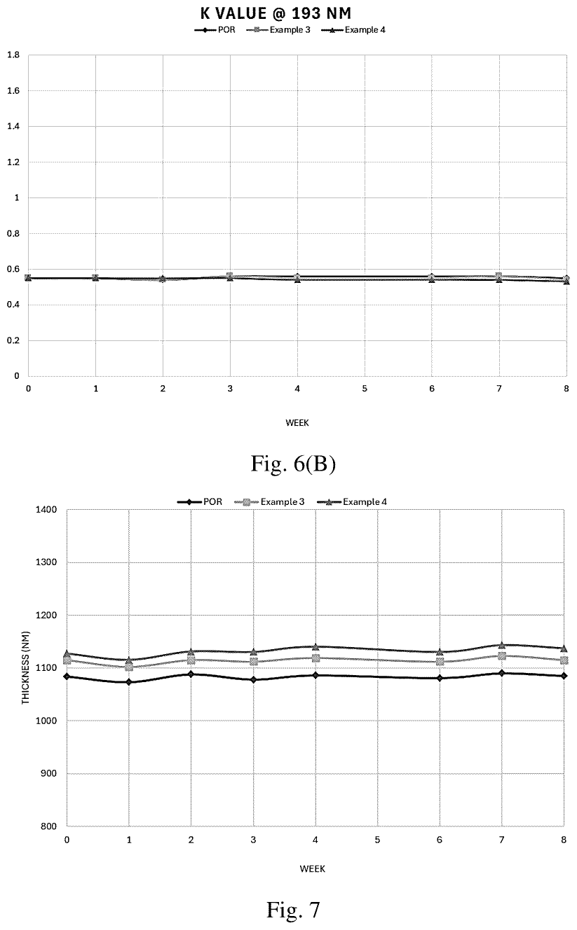

Materials were spin coated on 100-mm silicon wafers by spin coating the materials at 1,500 rpm and baking at 205° C. on a hot plate for 60 seconds. The baked films were compared to baseline every 7 days. FIGS. 6(A)-6(B) show that the optical constants remained consistent during the first 8 weeks of the aging study and were consistent with a POR spin-on carbon. FIG. 7 shows that the thickness also remained consistent during the 8 weeks of the aging study and were consistent with the material from Comparative Example 1 (“POR”).

### Example 9

### Coating Formulation

In this Example, 4.01 grams of a chromophore-grafted isocyanurate, 0.676 gram of PWL 1174, 0.081 gram of the modified CXC-1821 from Example 1, 0.0541 gram of tannic acid (Spectrum Chemical Mfg. Corp, Gardena, CA), 79.2 grams of PGMEA, and 16.8 grams of PGME were added to an Aicello bottle. The bottle was mixed on a roller for 24 hours and then was filtered using a 0.1 μm PTFE endpoint filter.

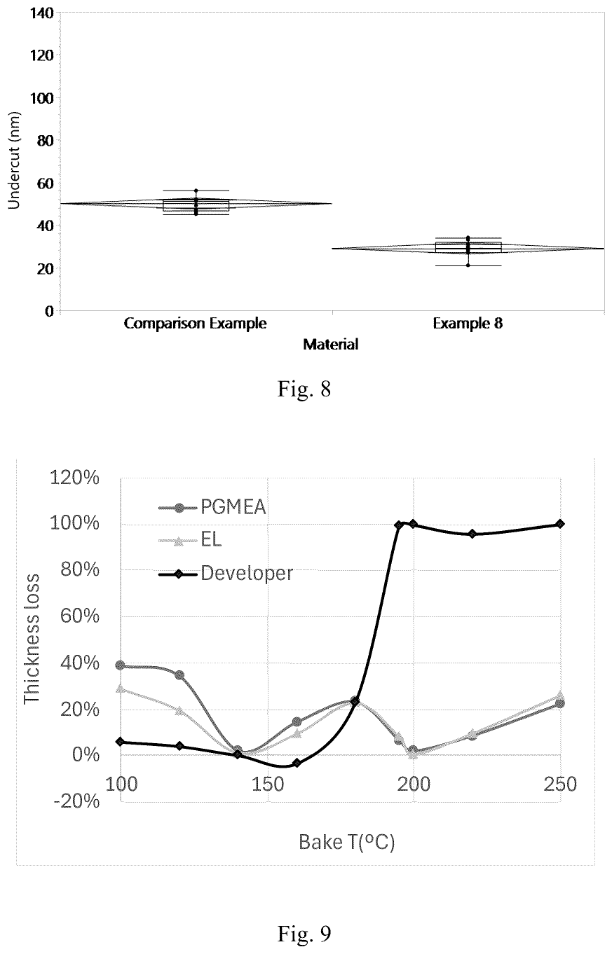

SC1 resistance testing was then performed by spin-coating an approximately 180-nm-thick coating of the material on top of a TiN liner topography substrate. The layer was then etched back with oxygen plasma to partially remove the material to mid-trench depth, and the substrate was immersed in an SC1 etchant bath at about 60° C. for about 100 seconds. An SEM (300 kx) cross-section analysis was performed to determine the undercut depth. When compared to the same composition utilizing TAG 2689 as the acid generator, the composition containing CXC-1821 generated similar etch and undercut results (FIG. 8).

### Example 10

### Develop-Back Coating Formulations

In this Example, two different batches of develop-back coating formulations were made using the method described in Example 3 of US Patent Publication 2025/0293030A1, except one used K-Pure® TAG 2689 as the acid generator, and the other used modified K-Pure® CXC-1821 (Example 1) as the acid generator.

Films of each material were spin coated on a 100-mm silicon wafer by spin coating at 1,500 rpm and then baked at various temperatures between 100° C. and 250° C. for 60 seconds. Thickness was then measured at 25 locations with a Gaertner Scientific Corporation model number 1SE-WS ellipsometer, and the average of those measurements was reported as the thickness. After thickness measurements were taken, films were puddled with PGMEA (stripped) for 20 seconds and then baked at 100° C. for 60 seconds. After a dehydration bake, the films were then measured with the ellipsometer, and the % film removal was calculated. This same procedure was repeated two more times but using ethyl lactate (EL) or a developer (PD523AD, 0.26N TMAH in water) instead of PGMEA.

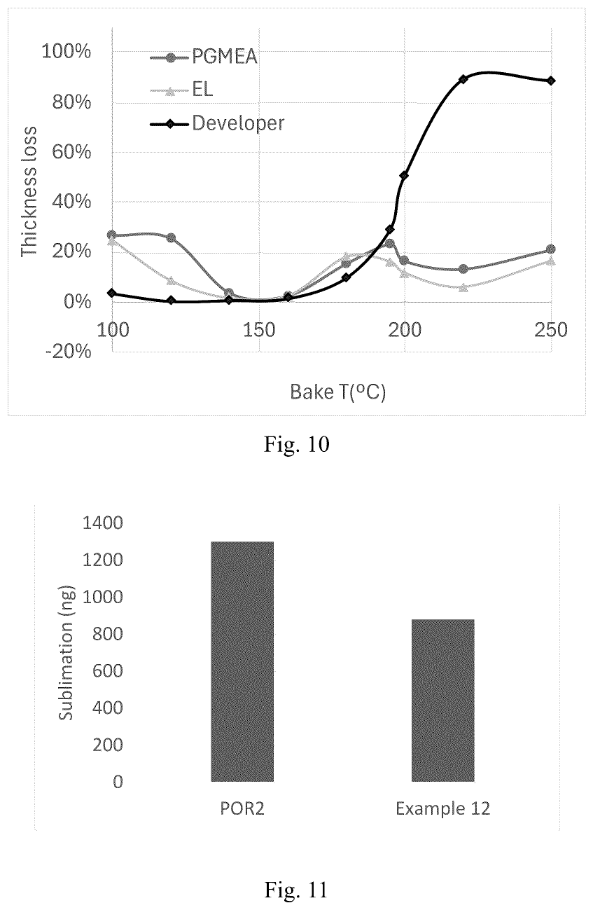

FIG. 9 shows the thickness loss of the material formulated with the TAG 2689, while FIG. 10 shows the thickness loss of the material formulated with the modified CXC-1821 of Example 1.

### Example 11

### Metal or Metalloid Ion Concentrations in CXC-1821 Material

In this Example, 1.50 grams of modified CXC-1821 obtained in Example 1 was dissolved in 98.50 grams of PGME. Additionally, 1.50 grams of unmodified (i.e., used “as obtained”) K-Pure® CXC-1821 was dissolved in 98.50 grams of PGME and used as a comparative sample. The respective metal or metalloid ion concentrations of each CXC-1821 solution were measured by inductively coupled plasma mass spectrometry (ICP-MS). The ICP-MS was an Agilent 8900 QQQ with a 1.0 mm torch diameter. The metal or metalloid ion concentrations are shown in Table 1. For most metal ions, the modified CXC-1821 solution showed significant reduction than the original solution, especially Li ion concentration.

TABLE 1
Metal or Metalloid Ion Concentrations in CXC-1821 Solution

| | 1.5% | MODIFIED 1.5% |
|---|---|---|
| | CXC-1821 | CXC-1821 |
| | IN PGME | IN PGME |
| METAL OR | REPORT | REPORT |
| METALLOID | CONC (PPB) | CONC (PPB) |
| Li | 40.076 | 0.400 |
| Be | 0.013 | <0.004 |
| Na | 7.627 | 0.747 |
| Mg | 1.721 | 0.040 |
| Al | 0.336 | <0.008 |
| K | 6.806 | 0.512 |
| Ca | 6.689 | 0.163 |
| Ti | 0.042 | <0.007 |
| V | 0.056 | 0.005 |
| Cr | 0.368 | 0.037 |
| Mn | 0.031 | <0.005 |
| Fe | 1.677 | 0.096 |
| Co | 0.104 | <0.008 |
| Ni | 0.062 | 0.012 |
| Cu | 0.077 | <0.006 |
| Zn | 0.975 | 0.017 |
| Ga | 0.121 | 0.012 |
| As | 0.109 | 0.015 |
| Rb | 0.016 | <0.006 |
| Sr | <0.007 | 0.001 |
| Zr | <0.103 | <0.005 |
| Nb | 0.013 | 0.006 |
| Mo | 0.060 | 0.008 |
| Ag | 0.309 | 0.058 |
| Cd | <0.021 | <0.006 |
| Sn | 0.064 | 0.109 |
| Cs | 0.010 | <0.004 |
| Ba | 0.026 | 0.001 |
| Hf | <0.046 | 0.002 |
| Ta | <0.056 | 0.003 |
| W | <0.164 | 0.008 |
| Re | 0.249 | <0.012 |
| Tl | <0.009 | 0.007 |
| Pb | 0.086 | 0.009 |
| Bi | 0.073 | 0.029 |

### Comparative Example 2

### Coating Formulation

Coating Formulation with Epoxy Cresol Novolac and Example 1 Modified Acid Generator

In this Example, 22.88 grams of ECN1299, 0.46 grams of a copolymer of glycidyl methacrylate and tert-butyl methacrylate, 0.04 gram of TAG-2689, 0.01 gram of Capstone™ FS-3100 surfactant, 137.53 grams of PGMEA, and 39.08 grams of PGME were added to an Aicello bottle. The bottle was mixed on a roller for 12 hours.

### Example 12

### Coating Formulation

Coating Formulation with Epoxy Cresol Novolac and Example 1 Modified Acid Generator

In this Example, 0.84 grams of ECN1299, 0.18 grams of a copolymer of glycidyl methacrylate and tert-butyl methacrylate, 0.034 gram of the modified CXC-1821 obtained in Example 1, 11.19 grams of PGMEA, and 2.46 grams of PGME were added to an Aicello bottle. The bottle was mixed on a roller for 12 hours.

### Example 13

Coat Quality Performance of Material from Example 12

Films were spin coated on a 100-mm silicon wafer by spin coating the material from Example 12 at 1,500 rpm and then placed under a QCM to determine the level of sublimation following the previously described procedure and parameters. Once under the microbalance, the films were exposed to a 205° C. hot plate for 120 seconds, and the sublimation level was recorded. These results were compared to the material from Comparative Example 2 (“POR2”). See FIG. 11.

### Example 14

Lithography evaluation of material from Example 12

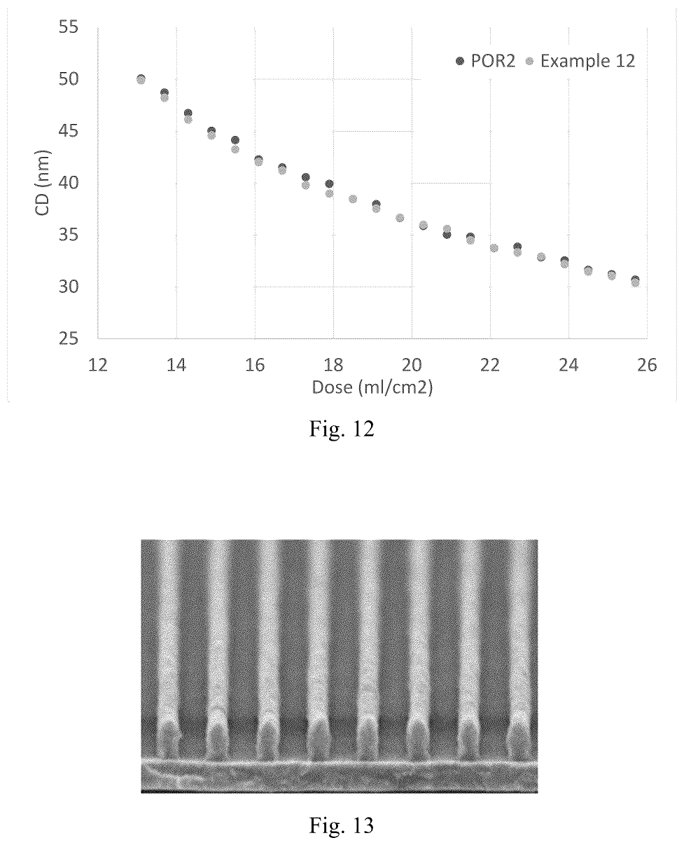

Material from Example 12 was coated on a blank 300-mm Si wafer by spin coating at 1,500 rpm and then were baked at 205° C. for 60 seconds. OptiStack® HM825 material (Brewer Science, Rolla, MO) was then spin coated at 1,500 rpm for 60 seconds on the SOC layer with a 205° C. bake for 60 seconds. A resist (AIM 5484, JSR, Tokyo, Japan) was then applied to the Si-HM layer and baked at 120° C. for 60 seconds. Lithography exposure at a wavelength of 193 nm was then performed, and development was carried out with OPD5262 (FUJIFILM Electronic Materials, Belgium) at imec. Process windows were then analyzed by top-down CD-SEM analysis and plotted (FIG. 12). The results were compared to the material from Comparative Example 2 (“POR2”). An SEM image (200 kX) of a wafer prepared in this Example is shown in FIG. 13, with that sample having a CD of 40 nm.

### Example 15

### Reaction of ECHTMS and 9-ACA

In this Example, 27.10 g of 2-(3,4-epoxycyclohexyl)ethyltrimethoxysilane (ECHTMS), 20.00 g of 9-anthracenecarboxylic acid (9-ACA), 0.55 g of benzyltriethylammonium chloride (BTEAC), and 250 g of PGME were weighed out in a 300-mL jacketed reactor. The reactor was equipped with reflux condenser and purged with nitrogen. The suspension was stirred at 400 rpm for 5 minutes. The temperature of the jacket was raised to 110° C. in 60 minutes. After that, the solution was stirred while the jacket was held at 110° C. for 24 hours. The reactor contents were allowed to cool and then downbottled into Aicello bottle. The bottle was stored in refrigeration.

The products of Example 15 were analyzed using gel permeation chromatography (GPC) measurement, which found the weight average Mw to be about 600-700 Da. The GPC spectrum was multimodal. High-performance liquid chromatography (HPLC) also indicated the presence of multiple products.

### Example 16

### High-TEOS Siloxane Polymer

In this Example, 0.81 g of 2-(3,4-epoxycyclohexyl)ethyltrimethoxysilane (ECHTMS), 5.23 g of phenyltrimethoxysilane (PTMS), 9.44 g of methyltrimethoxysilane (MTMS), and 48.12 g of tetraethoxysilane (TEOS) were added to 57 g of PGME in a three-necked round bottom flask. The flask was placed in an oil bath and equipped with a distillation set up. The whole reaction was purged with nitrogen, and then 29.70 g of 0.01M nitric acid was dropwise added into the reaction solution while it was stirred at 250 rpm at room temperature. After the addition of nitric acid, the temperature of the oil bath was gradually increased to 70° C. The reaction solution was held at 70° C. for seven hours, after which the reaction was allowed to cool to room temperature.

The cooled solution was transferred to a round bottom flask. The flask was placed on a rotavapor with water bath temperature set at 30° C. The residual byproducts (EtOH/MeOH/H₂O) were removed under reduced pressure (15 torr) using the rotavap. Finally, the concentrated solution was diluted with more PGME to restore the % solid of the solution to the same % it was before rotavap. The solution was transferred to an Aicello bottle, which was stored at −20° C.

The products were analyzed using GPC measurement, which found the weight average Mw to be about 2,000-2,500 Da. The GPC spectrum was monomodal.

### Example 17

Coating Formulation with Siloxane Polymer and Example 1

In this Example, 6.06 g of the product of Example 16 (solid %˜16.75% in 23.44% of PGME and 76.56% of PGMEA) and 4.51 g of the product of Example 15 (solid %˜18.39% in 100% of PGME) were weighed out in a 100-ml Aicello bottle. Then, 3.69 g of maleic acid solution (solid %˜0.5% in 100% of PGME) and 0.74 g of modified CXC-1821 solution obtained from Example 1 (solid %˜5% in 100% of PGME) were also added to the same bottle. Finally, 59.43 g of PGME, 9.81 g of water, and 15.76 g of PGMEA were weighed out in the same bottle to dilute the formulation. The resulting formulation was left on a roller for about 60 minutes to assure homogeneous mixing. The solution was purged through a 0.1-μm filter. The filtered solution was transferred to a 100-ml Aicello bottle, which was stored in refrigeration. The solution was applied to a 100-mm bare silicon wafer by spin coating at 1,500 rpm for 60 seconds. The film was then baked at 205° C. for 60 seconds. The thickness of the film was about 50 nm after baking. The film was resistant to both organic solvent and resist developer (PD523). The n and k values at 248 nm were about 1.47 and 0.27, respectively.

### Example 18

### Lithography Evaluation of Example 17 Material

The material of Example 17 was used to form silicon hardmasks on top of a spin-on carbon material and under a photoresist in a trilayer stack and tested for DUV lithography and pattern transfer. In this procedure, DUV 252-330 material (Brewer Science, Inc.) was spun on a bare silicon wafer and baked (Layer 1), and then the SiHM-forming material of Example 17 was spun onto the baked SOC layer and baked (Layer 2). Table 2 below provides processing conditions used to form Layers 1 and 2.

TABLE 2
Processing Conditions for Formation of SOC and SiHM Layers

| Layer 1 | | | | Layer 2 | | | |
|---|---|---|---|---|---|---|---|
| | | | Target | | | | Target |
| | Spin | Bake | Thickness | | Spin | Bake | Thickness |
| SOC | (rpm/s) | (° C./s) | (nm) | SiHM | (rpm/s) | (° C./s) | (nm) |
| DUV 252-330 | 880/30 | 205/60 | 400 | Example 17 | 1382/30 | 205/60 | 50 |
| | | | | Material | | | |

Next, a commercially available KrF photoresist (M91Y-9 cP, JSR) was spun on top of the formed SiHM, and a DUV lithography exposure and pattern transfer test was conducted according to the conditions set forth in Table 3.

TABLE 3
Processing Conditions for Lithography Test

| Parameter | Conditions | Notes |
|---|---|---|
| Resist: | M91Y-9cP | |
| Resist thickness (nm): | 420 | |
| Resist coat (rpm/s): | 2734/40 | |
| Target CD (nm/pitch): | 160 L/300 P | MCD150 L/300 P*** |
| PAB (° C./s): | 130/90 | |
| PEB (° C./s): | 130/90 | |
| Illumination mode: | Annular | |
| NA: | 0.70 | |
| Sigma (outer/inner): | 0.70/0.35 | |
| Center dose/step | 15/0.8 | |
| (mJ/cm²): | | |
| Focus offset/step (μm): | 0.05/0.1 | |
| Developer type/time (s): | OPD262**/60 | |

* Available from JSR.
**Available from Fujifilm.
***Mask CD = 150 nm line but overexposed, so the resulting features had 160-nm lines.

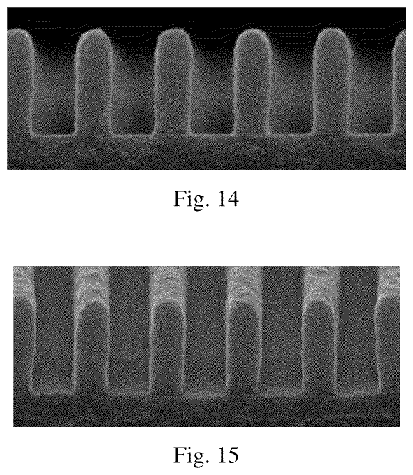

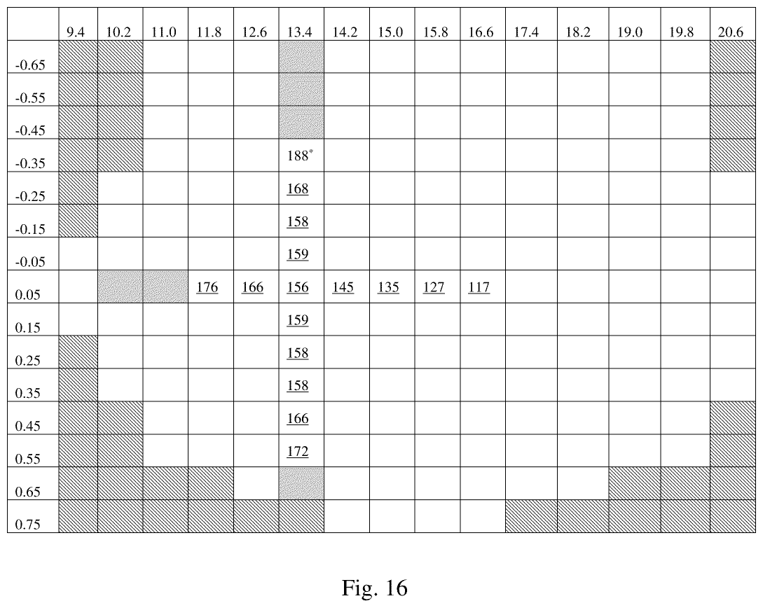

After the pattern had been developed in the resist as described above, scanning electron micrographs of the sectioned trilayer stack were taken at tilt angles of 90 and 75 degrees (FIGS. 14 and 15, respectively). The focus-exposure matrix (FEM) is shown in FIG. 16. The underlined numbers designate nicely formed features, while the asterisk (*) at “188*” designates the appearance of bridging.

1. A method of forming a structure, said method comprising applying a composition on a surface of a substrate, or on one or more intermediate layers optionally present on said surface of said substrate, to form a PFAS-free layer, said composition comprising a compound and an acid generator source dispersed or dissolved in a solvent system, wherein:
said compound is polymeric, oligomeric, or monomeric;
said acid generator source comprises a quaternary ammonium catalyst, and a 1.5% solution of said acid generator source comprises less than about 300 ppb metal ions; and
said wherein PFAS-free layer comprises less than about 0.05% by weight PFAS, wherein at least one of (I), (II), (III), (IV), or (V) is true:
(I) said PFAS-free layer comprises about 50% by weight to about 99% by weight carbon, and further comprising:
forming a hardmask layer on said PFAS-free layer;
optionally forming one or more additional intermediate layers on said hardmask layer; and
forming a photoresist layer on said one or more additional intermediate layers on said hardmask layer, if present, or on said hardmask layer if no additional intermediate layer is present;
(II) one or more intermediate layers is present and includes an uppermost intermediate layer that comprises a carbon-rich layer that comprises about 50% by weight to about 99% by weight carbon, and further comprising:
optionally forming one or more additional intermediate layers on said PFAS-free layer; and
forming a photoresist layer on said one or more additional intermediate layers on said PFAS-free layer, if present, or on said PFAS-free layer if no additional intermediate layer is present;
(III) further comprising:
(b) (i) forming a carbon-rich layer that comprises about 50% by weight to about 99% by weight carbon on said PFAS-free layer;
(ii) optionally forming one or more additional intermediate layers on said carbon-rich layer; and
(iii) forming a photoresist layer on said one or more additional intermediate layers on said carbon-rich layer, if present, or on said carbon-rich layer if no additional intermediate layer is present;
(b) (i) forming a hardmask layer on said PFAS-free layer;
(ii) optionally forming one or more additional intermediate layers on said hardmask layer; and
(iii) forming a photoresist layer on said one or more additional intermediate layers on said hardmask layer, if present, or on said hardmask layer if no additional intermediate layer is present; or
(c) (i) forming a carbon-rich layer that comprises about 50% by weight to about 99% by weight carbon on said PFAS-free layer;
(ii) forming a hardmask layer on said carbon-rich layer;
(iii) optionally forming one or more additional intermediate layers on said hardmask layer; and
(iv) forming a photoresist layer on said one or more additional intermediate layers on said hardmask layer, if present, or on said hardmask layer if no additional intermediate layer is present;
(IV) said surface of said substrate comprises a pattern comprising a plurality of gaps, and said applying a composition comprises depositing said composition in at least some of said gaps; or
(V) said one or more intermediate layers are present and include one or both of a carbon-rich layer that comprises about 50% by weight to about 99% by weight carbon or a hardmask layer, and further comprising forming a photoresist layer on said PFAS-free layer.

2. The method of claim 1, wherein said quaternary ammonium catalyst is a blocked superacid catalyst.

3. The method of claim 1, wherein said quaternary ammonium catalyst is a salt whose cationic portion comprises 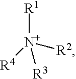
where each of R¹ to R⁴ is individually chosen from alkyls and aryls, with at least one of R¹ to R⁴ comprising an aryl comprising 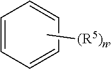
where each R⁵ is individually chosen from —H, alkyls, or alkoxys.

4. The method of claim 1, wherein said quaternary ammonium catalyst is a salt whose cationic portion comprises

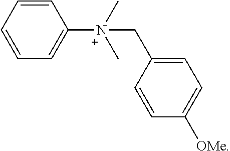 [SMILES: COc1ccc(C[N+](C)(C)c2ccccc2)cc1]

5. The method of claim 1, wherein said quaternary ammonium catalyst comprises

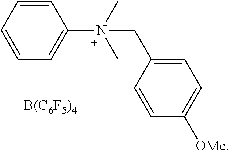 [SMILES: "B(C6F5)4" ; COc1ccc(C[N+](C)(C)c2ccccc2)cc1]

6. The method of claim 1, wherein said compound is chosen from one or more of:
polymers and/or oligomers comprising monomers chosen from phenolic compounds, styrene, styrene-containing compounds, glycidyl-containing compounds, 2-hydroxyethyl methacrylate, hydroxypropyl methacrylate, hydroxybutylacrylate, silanes, siloxanes, or combinations thereof;
polymers chosen from polystyrene, functionalized polystyrene derivatives, polysulfones, polyethersulfones, poly(ether ether ketone), poly(vinyl naphthalene)), polycarbonates, epoxies, novolacs, polyimides, or combinations thereof; or
phenolic compounds, styrene, styrene-containing compounds, glycidyl-containing compounds, 2-hydroxyethyl methacrylate, hydroxypropyl methacrylate, hydroxybutylacrylate, silanes, or combinations thereof.

7. The method of claim 1, wherein said composition does not include any acid generators or catalysts other than said quaternary ammonium catalyst.

8. The method of claim 1, wherein said substrate is formed from one or more of silicon, SiGe, SiO₂, Si₃N₄, SiON, aluminum, tungsten, tungsten silicide, gallium arsenide, germanium, tantalum, tantalum nitride, Ti₃N₄, hafnium, HfO₂, ruthenium, indium phosphide, tetramethyl silate and tetramethylcyclotetrasiloxane combinations, SiCOH, or glass.

9. The method of claim 1, further comprising:
exposing said photoresist layer to radiation so as to form a pattern in said photoresist layer; and
transferring said pattern to said substrate.

10. The method of claim 9, wherein said exposing is carried out at a wavelength chosen from about 193 nm or about 13.5 nm.

11. The method of claim 1, wherein said gaps of (IV) are formed from topographic features, and said composition is applied at a sufficient thickness to cover said topographic features and form a planarizing layer of said PFAS-free layer on said topographic features.

12. The method of claim 1, wherein at least (I) is true.

13. A method comprising:
storing a dispersion or solution of an acid generator source comprising an acid generator, said storing being carried out at a temperature of about −20° C. to about 25° C. for about 32 hours to about 72 hours so as to generate a precipitate including said acid generator; and
collecting said precipitate.

14. The method of claim 13, wherein said acid generator comprises a blocked superacid catalyst.

15. The method of claim 13, wherein said acid generator comprises a quaternary ammonium catalyst.

16. The method of claim 15, wherein said quaternary ammonium catalyst is a salt whose cationic portion comprises 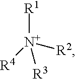
where each of R¹ to R⁴ is individually chosen from alkyls and aryls, with at least one of R¹ to R⁴ comprising an aryl comprising 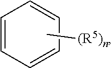
where each R⁵ is individually chosen from —H, alkyls, or alkoxys.

17. The method of claim 15, wherein said quaternary ammonium catalyst is a salt whose cationic portion comprises

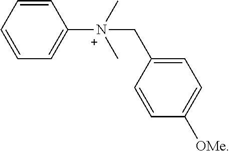 [SMILES: COc1ccc(C[N+](C)(C)c2ccccc2)cc1]

18. The method of claim 15, wherein said quaternary ammonium catalyst comprises

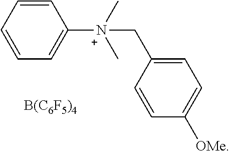 [SMILES: "B(C6F5)4" ; COc1ccc(C[N+](C)(C)c2ccccc2)cc1]

19. The method of claim 13, wherein said acid generator source comprises a particulate material, and further comprising mixing said particulate material with a solvent for about 4 hours or more to form said dispersion or solution.

20. The method of claim 13, wherein said acid generator source further comprises deionized water.

21. The method of claim 19, wherein said solvent comprises at least one of propylene glycol monomethyl ether acetate, propylene glycol monomethyl ether, propylene glycol ethyl ether, acetone, ethyl lactate, propylene carbonate, deionized water, cyclohexanone, γ-butyrolactone, cyclopentanone, or mixtures thereof.

22. The method of claim 13, wherein one or both of the following is true:
(a) said acid generator source comprises less than 0.05% by weight PFAS; or
(b) said acid generator source comprises less than 0.05% by weight antimony.

23. The method of claim 13, wherein one or both of the following is true:
(a) a 1.5% solution of said precipitate comprises less than about 300 ppb metal ions; or
(b) a 1.5% solution of said precipitate comprises 75% or less metal ions as compared to a 1.5% solution of said acid generator source.
